(19) United States
(12) Patent Application Publication
(10) Pub. No.: US 2014/0235939 A1
(43) Pub. Date: Aug. 21, 2014

(54) METHOD FOR PRODUCING AN ENDOSCOPE AND AN ENDOSCOPE

(71) Applicant: OLYMPUS WINTER & IBE GMBH, Hamburg (DE)

(72) Inventor: Uwe SCHOELER, Hoisdorf (DE)

(73) Assignee: OLYMPUS WINTER & IBE GMBH, Hamburg (DE)

(21) Appl. No.: 14/182,463

(22) Filed: Feb. 18, 2014

(30) Foreign Application Priority Data
Feb. 18, 2013 (DE) 10 2013 202 539.4

(51) Int. Cl. A61B 1/00

(52) U.S. Cl. CPC A61B 1/0011; A61B 1/00064
USPC 600/101; 29/428

(57) ABSTRACT

A method for producing an endoscope with a distal end and a proximal end, wherein a tube is provided, which has an opening for receiving a window. The method including, inserting a window into the opening of the tube, permanently fixing the window in the opening of the tube, and galvanically applying a noble metal to the tube in the area of the window and around the window. Also provided is an endoscope with a distal end and a proximal end, wherein the endoscope includes a tube, which extends up to the distal end, wherein a window is inserted into the tube on the distal end, which is permanently fixed in the tube by means of a hermetically sealing material. Wherein the tube on the distal end and the hermetically sealing material are coated on the outside with a noble metal.

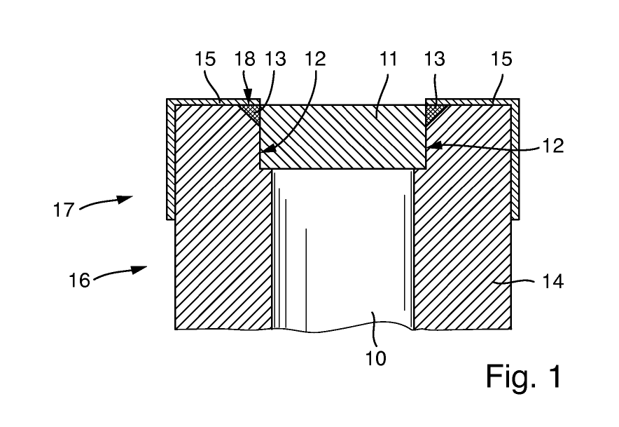

### CROSS-REFERENCE TO RELATED APPLICATION

The present application is based upon and claims the benefit to DE 10 2013 202 539.4 filed on Feb. 18, 2013, the entire contents of which is incorporated herein by reference.

### BACKGROUND

1. Field

The invention relates to a method for producing an endoscope with a distal end and a proximal end, wherein a tube is provided, which has an opening for receiving a window. Furthermore, the invention relates to an endoscope with a distal end and a proximal end, wherein the endoscope comprises a tube, which extends up to the distal end, wherein a window is inserted into the tube on the distal end, which is permanently fixed in the tube by means of a hermetically sealing material.

2. Prior Art

Endoscopes normally have a window on their distal end in order to be able to look at objects to be observed on the distal end or near the distal end of the endoscope. Inside the endoscope in the area of the window, i.e. on the distal end, a camera can be provided, for example, which can take pictures through a corresponding window, which is provided on the distal end of the endoscope. Alternatively, optical fibres and, if applicable, optics can be provided in order to direct pictures from the distal end of the endoscope to the proximal end.

In the case of endoscopes used in medical technology or respectively in surgery, it is necessary that the endoscopes can be sufficiently cleaned. For this, they must be autoclavable, dishwasher-safe and also suitable for being stable in an aggressive chemical environment. A window in an endoscope must thus be installed in a hermetically sealing manner. For this, it is known to metalize the side walls of the window that border the tube material after installation of the window into the tube in a multi-step process in order to then subsequently solder them. This is relatively complex. Moreover, it is required to use a solder, which will not be corroded in particular when using corrosive chemicals to clean the endoscope.

### SUMMARY

An object of the present invention is to specify a method for producing an endoscope that enables an insertion of a window in the distal end of the endoscope, which is less complex and nonetheless leads to an autoclavable, dishwasher-safe and chemically stable endoscope.

Accordingly, the object of the invention is to specify such an autoclavable, dishwasher-safe and chemically stable endoscope.

The object is solved through a method for producing an endoscope with a distal end and a proximal end, wherein a tube is provided, which has an opening for receiving a window, with the following process steps:
Insertion of a window into the opening of the tube,
Permanent fixation of the window in the opening of the tube,
Galvanic application of a noble metal to the tube in the area of the window and around the window.

Through the galvanic application of a noble metal to the tube in the area of the window and around the window, the endoscope is strengthened in the area of the opening so that it can be autoclaved, is dishwasher safe and resistant to aggressive chemicals, since the noble metal also covers in particular a connecting means or connecting material of the window to the tube. Correspondingly, corrosive chemicals cannot hereby corrode the connecting material. Within the framework of the invention, ‘around the window’ also means adjacent to the window. For the galvanic application of a noble metal to the tube in the area of the window and around the window, the tube is preferably immersed in a galvanic bath. The window can be installed on the distal front side and/or in the distal surface shell of the tube.

During the step of the galvanic application of the noble metal, the window is preferably fixed in the opening of the tube. The window is thus also immersed in the galvanic bath during the galvanic application of the noble metal to the tube in the area of the window and around the window.

For the case when a connecting material is provided between the window and the tube, it is also coated with the noble metal. The connecting material is preferably hermetically sealing. The connecting material can be an adhesive or a solder.

A method according to the invention is particularly simple when the permanent fixation is bonding by an adhesive. It is in particular preferable if an adhesive is hereby used that is electroconductive. When using an adhesive that is electroconductive, the noble metal also precipitates on the adhesive in the galvanic bath so that the adhesive is also resistant to chemicals after the noble metal coating. The surface of the adhesive, in particular after the bonding by an adhesive, is preferably post-processed in order to increase the conductivity. During post-processing, a laser beam with a corresponding energy density can for example be directed at the surface of the adhesive or respectively glue so that the adhesive content of the glue evaporates accordingly on the surface and accordingly more metal powder remains on the surface for example during the provision of a glue with metal powder. Correspondingly more metal particles are hereby released on the surface so that a closed metal layer is created during the galvanic application of the noble metal, which protects the underlying adhesive during autoclaving and also in particular during processing in a corrosive atmosphere, i.e. during the use of corresponding chemicals.

The permanent fixation is preferably a soldering, in particular a hard-soldering or an active soldering. This has the advantage that the connecting material, i.e. the material that connects the window with the tube, is electroconductive. The surface of the solder can also be post-processed correspondingly in order to remove non-conductive islands potentially created on the surface. A laser beam treatment such as a laser ablation can be provided for this.

Particularly preferred is a method in which a joining partner, in particular the distal end of the tube, is made of an electroconductive material with an expansion coefficient that corresponds with the expansion coefficient of the window.

The joining partner is preferably made of Kovar. At least the distal end of the tube is preferably made of Kovar. In general, the entire tube can also be made of Kovar. Moreover, the window is also preferably sapphire (Al₇O₃) Kovar can be for example an alloy of 54% iron, 29% nickel and 17% cobalt.

Furthermore, the object is solved through an endoscope with a distal end and a proximal end, wherein the endoscope comprises a tube that extends up to the distal end, wherein a window is installed in the tube on the distal end, which is permanently fixed in the tube by means of a hermetically sealing material, further characterized in that the tube on the distal end and the hermetically sealing material are coated on the outside with a noble metal.

The noble metal coating is preferably provided adjacently up to the window. The noble metal can be gold or platinum or another noble metal.

The hermetically sealing material is preferably an adhesive or a solder. A special hermetic seal or respectively a particularly efficient connection of the window with the tube is hereby possible. The opening is preferably large enough so that a tight clearance fit of the window in the opening is provided, namely at approximately one fourth the height to at least one half the height of the window in the tube. For the method for producing the endoscope, this means that preferably the window is first inserted in a tight clearance fit, in which the window is already held and the remainder is then soldered or glued.

The endoscope according to the invention is in particular preferred when the adhesive is electroconductive. A noble metal can hereby be applied in a particularly simple manner and in particular also on the adhesive. The adhesive is preferably filled with metal powder. Electrical conductivity can hereby be established in a very simple manner. A very even and closed noble metal coating on the surface, i.e. in particular on the front side of the endoscope, then results when the surface of the adhesive is processed so that the surface has a higher electrical conductivity than the volume of the adhesive.

As an alternative to the adhesive, a solder can be provided that is preferably a silver-copper solder or a gold-tin solder. Correspondingly, this solder can also be coated with a noble metal layer.

At least the distal end of the tube is preferably made of an electroconductive material that has an expansion coefficient that corresponds with that of the window. Thermal stress is hereby considerably reduced or respectively excluded. The electroconductive material is preferably Kovar. Furthermore, the window is preferably made of sapphire.

According to the invention, it is suggested to modify the distal end of an endoscope. For this, a window consisting of a sapphire disc is inserted into a tight clearance fit of a tube up to approximately one fourth the height or up to approximately half the height. A conical joining gap remaining towards the distal end or respectively towards the surface is filled for example with adhesive. The adhesive should be selected so that it has a certain residual viscosity and/or flexibility after hardening in order to be able to receive stress during autoclaving. In order to achieve CDS (Cleaning Disinfection Sterilization) durability of the adhesive and also in order to prevent the adhesive from absorbing moisture, it is suggested to fill the adhesive with metal powder and to then apply a noble metal coating. After hardening, the adhesive surface is preferably processed, for example by means of laser beams, in order to expose a sufficiently large amount of metal particles on the surface. The surface of the adhesive can also be processed for example through polishing.

During galvanic coating, these metal particles serve to achieve a most closed possible coating with a noble metal in the area of the adhesive. The proximal end of the tube of the endoscope or respectively the proximal end of the endoscope is provided with a closed noble metal coating, for example a gold coating, which protect the underlying adhesive and the underlying tube during sterilization or respectively disinfection, even in particular with corrosive chemicals.

An active soldering process with a solder can be used instead of an adhesive. In order to avoid stress fractures during this process, it is suggested to use a Kovar for at least one metallic joining partner, for example the distal end of the tube, i.e. a metal with a thermal expansion coefficient that is very similar to that of the window, for example sapphire. It is then suggested to seal or respectively provide this material together with the solder, which can be a silver-copper solder for example, with a noble metal in order to protect these materials during the reprocessing process of the endoscope, i.e. during sterilization, autoclaving and/or exposure to chemical substances.

Further characteristics of the invention will become evident from the description of the embodiments according to the invention together with the claims and the attached drawings. Embodiments according to the invention can fulfil individual characteristics or a combination of several characteristics.

### BRIEF DESCRIPTION OF THE DRAWINGS

The invention is described below, without restricting the general idea of the invention, based on exemplary embodiments in reference to the drawings, whereby we expressly refer to the drawings with regard to the disclosure of all details according to the invention that are not explained in greater detail in the text. It shows in the FIGURE, which is a schematic cross-sectional view through a distal end of an endoscope according to the invention.

### DETAILED DESCRIPTION

The FIGURE shows a schematic cross-sectional view through a distal end 17 of an endoscope 16. A corresponding tube 10 is represented with a tube wall 14. A window 11, for example made of sapphire, is installed in the tube wall 14. For this, a clearance fit 12 is first provided in the tube wall 14, in which the sapphire window 11 or respectively window 11 is pressed. In this exemplary embodiment, the clearance fit 12 is designed approximately over half the height of the window 11. Moreover, a conical joint gap is provided in the upper area of the tube 10 towards the inside, into which an adhesive 13 is inserted, which is metallically conductive. The surface 18 of the adhesive 13 is preferably processed so that the conductivity there is higher than in the volume of the adhesive 13.

Moreover, a gold coating 15 is shown, which can also be a platinum coating and is applied on the distal end of the tube to the tube 10 or respectively the tube wall 14 from the outside and also on the front side of the tube. Moreover, the gold coating 15 extends beyond the surface 18 of the adhesive 13 and up to the edge of the sapphire window 11.

Instead of a connection by means of an adhesive 13, a solder can also be used. In the case of the use of a solder, it is very useful to make the tube 10 or respectively a distal section of the tube 10 from for example Kovar. The entire area made of this material should then be coated accordingly with the gold coating 15 or another noble metal coating. Moreover, the solder that can be arranged originating like the adhesive 13 in the FIGURE should also be covered with the gold coating 15 or respectively the noble metal coating.

All named characteristics, including those taken from the drawing alone, and individual characteristics, which are disclosed in combination with other characteristics, are considered alone and in combination as essential for the invention. Embodiments according to the invention can be fulfilled through individual characteristics or a combination of several characteristics.

### LIST OF REFERENCES

10 Tube
11 Window
12 Clearance fit
13 Adhesive
14 Tube wall
15 Gold coating
16 Endoscope
17 Distal end
18 Surface

What is claimed is:

1. A method for producing an endoscope with a distal end and a proximal end, wherein a tube is provided, which has an opening for receiving a window, the method comprising:
inserting a window into the opening of the tube;
permanently fixing the window in the opening of the tube; and
galvanically applying a noble metal to the tube in the area of the window and around the window.

2. The method according to claim 1, wherein the window is fixed in the opening of the tube during the galvanic application of the noble metal.

3. The method according to claim 1, wherein the permanent fixation is bonding by an adhesive.

4. The method according to claim 3, wherein the adhesive is electroconductive.

5. The method according to claim 4, wherein a surface of the adhesive is post-processed to increase conductivity.

6. The method according to claim 4, wherein the post-processing is after the bonding by the adhesive.

7. The method according to claim 1, wherein the permanent fixation is a soldering.

8. The method according to claim 7, wherein the soldering is one of a hard soldering or an active soldering.

9. The method according to claim 7, wherein the galvanic application of the noble metal comprises using a joining partner made of an electroconductive material with an expansion coefficient that corresponds with that of the window.

10. The method according to claim 9, wherein the joining partner comprises a distal end of the tube.

11. The method according to claim 9, wherein the joining partner is made of Kovar.

12. An endoscope with a distal end and a proximal end, wherein the endoscope comprises:
a tube extending to the distal end;
a window inserted in the tube on the distal end;
a hermetically sealing material for permanently fixing the window in the tube;
wherein the tube on the distal end and the hermetically sealing material are coated on an outside with a noble metal.

13. The endoscope according to claim 12, wherein the hermetically sealing material is one of an adhesive or a solder.

14. The endoscope according to claim 13, wherein the adhesive is electroconductive.

15. The endoscope according to claim 14, wherein the adhesive contains metal powder.

16. The endoscope according to claim 14, wherein a surface of the adhesive is processed such that the surface has a higher electrical conductivity than a volume of the adhesive.

17. The endoscope according to claim 13, wherein the solder is one of a silver-copper solder or a gold-tin solder.

18. The endoscope according to claim 12, wherein at least a distal end of the tube is made of an electroconductive material having an expansion coefficient corresponding with an expansion coefficient of the window.

19. The endoscope according to claim 18, wherein the electroconductive material is Kovar.

20. The endoscope according to claim 12, wherein the window is made of sapphire.
